(19) United States
(12) Patent Application Publication
(10) Pub. No.: US 2023/0320670 A1
(43) Pub. Date: Oct. 12, 2023

(54) METHOD AND SYSTEM FOR PREDICTING GERIATRIC SYNDROMES USING FOOT CHARACTERISTICS AND BALANCE CHARACTERISTICS

(71) Applicant: KOREA INSTITUTE OF SCIENCE AND TECHNOLOGY, Seoul (KR)

(72) Inventors: Kyung-Ryoul MUN, Seoul (KR); Jinwook KIM, Seoul (KR); Dawoon JUNG, Seoul (KR)

(73) Assignee: KOREA INSTITUTE OF SCIENCE AND TECHNOLOGY, Seoul (KR)

(21) Appl. No.: 17/716,220

(22) Filed: Apr. 8, 2022

(51) Int. Cl. A61B 5/00; G06T 7/00; A61B 5/103

(52) U.S. Cl. CPC A61B 5/7275; G06T 7/0012; A61B 5/4023; A61B 5/1038; A61B 5/7267; A61B 5/0064; A61B 5/702; G06T 2207/10028; G06T 2207/30004; G06T 2207/30196; G06T 2207/20081; G06T 2207/20084

(57) ABSTRACT

The method and system for predicting geriatric syndrome according to the present invention predicts a risk degree of geriatric syndrome of a subject based on the foot depth image and plantar pressure data acquiring the foot depth image and the plantar pressure data.

### BACKGROUND OF THE INVENTION

### Field of the Invention

The present invention relates to a method and system for predicting geriatric syndrome, and more particularly, to a method and system for predicting geriatric syndrome using foot characteristic information and balance characteristic information.

### Description of the Related Art

With the rapid aging of the population, various geriatric syndromes are occurring. These syndromes typically include frailty, cognitive impairment, sarcopenia, and depression. Frailty is defined as a condition of decreased physiologic reserve due to the deterioration of overall function due to aging that leads to a vulnerable to external stressors and a reduced ability to maintain homeostasis. Cognitive impairment is defined as a condition in which memory, attention, language ability, spatiotemporal ability, judgment, etc. are deteriorated, and corresponds to the early stage of dementia. Sarcopenia is a condition in which muscle mass and muscle function are lost above a certain level, and if sarcopenia is left unattended, falls, fractures, metabolic syndrome, second diabetes, depression, etc. may occur. Depression, which is known to affect one in three elderly Koreans, is easily deemed as a simple aging phenomenon because the main symptoms are memory loss, anorexia, lethargy, insomnia and anxiety, headache and joint pain. However, depression in the elderly is more likely to lead to suicide, so early diagnosis and appropriate treatment are necessary.

If these geriatric syndromes are not diagnosed at an early stage, they cause various complications, increasing the personal and social burden of medical expenses. However, currently, an experienced specialist is required for an accurate diagnosis of geriatric syndrome. In addition, because of the need for a clinical environment with special facilities and equipment and clinicians, as well as a complex and time-consuming examination process, many elderly people are not diagnosed with geriatric syndromes at an early stage.

Therefore, there is a need for a method and system capable of predicting geriatric syndromes without expensive equipment, skilled experts, and complicated examination processes.

### SUMMARY OF THE INVENTION

The present invention has been devised to solve the above problems. Specifically, the present invention relates to a method and system for predicting geriatric syndrome using foot characteristic information and balance characteristic information.

A system for predicting geriatric syndrome according to one embodiment of the present specification includes a data acquisitor that acquires a foot depth image and plantar pressure data of a subject; a foot characteristic information generator that generates foot characteristic information of the subject with the foot depth image obtained in a state in which the subject's posture is stable; a gait characteristic information generator that generates gait characteristic information of the subject based on the foot characteristic information of the subject by using a first learning model trained to output the gait characteristic information based on the foot characteristic information; a balance characteristic information generator that generates balance characteristic information of the subject with the plantar pressure data obtained in a state in which the subject's posture is unstable; and a geriatric syndrome predictor that predicts a risk degree of geriatric syndrome of the subject based on the foot characteristic information of the subject, the gait characteristic information of the subject and the balance characteristic information of the subject, by using a second learning model trained to output the risk degree of geriatric syndrome based on the foot characteristic information, the gait characteristic information and the balance characteristic information.

A method for predicting geriatric syndrome according to another embodiment of the present specification includes the steps of acquiring a foot depth image in a state in which a subject's posture is stable, and plantar pressure data in a state in which the subject's posture is unstable; generating foot characteristic information of the subject with the foot depth image; generating gait characteristic information of the subject based on the foot characteristic information of the subject by using a first learning model trained to output the gait characteristic information based on the foot characteristic information; generating balance characteristic information of the subject with the plantar pressure data; and predicting a risk degree of geriatric syndrome of the subject based on the foot characteristic information of the subject, the gait characteristic information of the subject and the balance characteristic information of the subject, by using a second learning model trained to output the risk degree of geriatric syndrome based on the foot characteristic information, the gait characteristic information and the balance characteristic information.

The system and method for predicting geriatric syndrome according to an embodiment of the present invention can predict a risk degree of geriatric syndrome of a subject only by a test for acquiring simple foot characteristic information and simple balance characteristic information.

In other words, the present invention can predict a risk degree of geriatric syndrome and provide it to a subject without expensive equipment, skilled experts, and complicated examination process, and the present invention can provide a subject with an opportunity for early diagnosis of related diseases, management of symptoms, and appropriate treatment.

### BRIEF DESCRIPTION OF THE DRAWINGS

FIG. 1 is a block diagram of a system for predicting geriatric syndrome according to an embodiment of the present invention.

FIG. 2 is an exemplary diagram of a data acquisitor.

FIG. 3 shows an example of an unstable posture taken by a subject when plantar pressure data is acquired.

FIGS. 4A and 4B are exemplary diagrams illustrating a foot depth image and foot characteristic information obtained therefrom.

FIG. 5 is an exemplary diagram for explaining gait characteristic information.

FIG. 6 is an exemplary diagram illustrating a change in the center of plantar pressure.

FIG. 7 is a flowchart of a method for predicting geriatric syndrome using foot characteristic information according to an embodiment of the present invention.

### DETAILED DESCRIPTION OF THE INVENTION

Hereinafter, preferred embodiments according to the present invention will be described in detail with reference to the accompanying drawings. The detailed description set forth below in conjunction with the appended drawings is intended to describe exemplary embodiments of the present invention and is not intended to represent the only embodiments in which the present invention may be implemented. The following detailed description includes specific details in order to provide a thorough understanding of the present invention. However, one skilled in the art will recognize that the present invention may be practiced without these specific details. Specific terms used in the following description are provided to help the understanding of the present invention, and the use of these specific terms may be changed to other forms without departing from the technical spirit of the present invention.

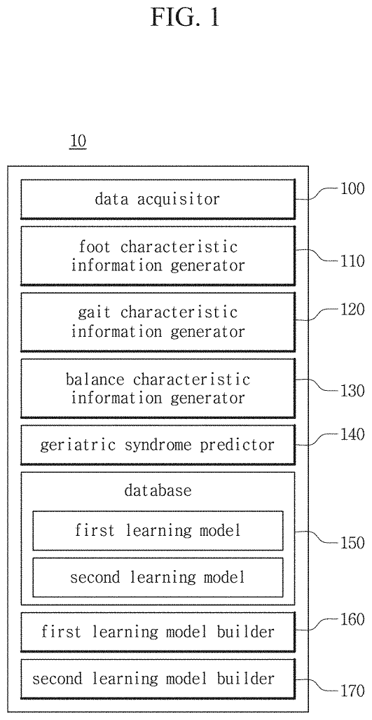

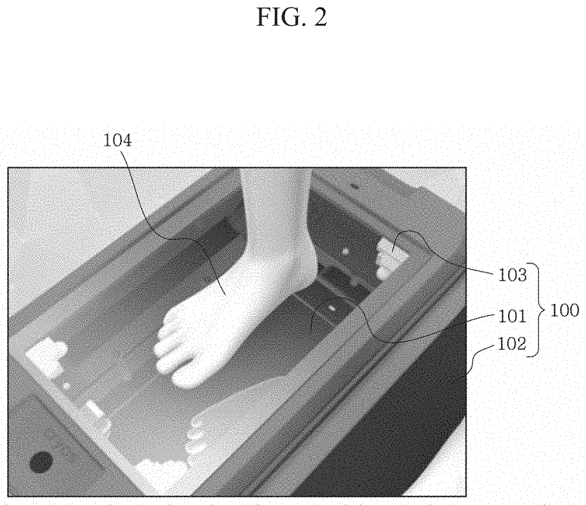

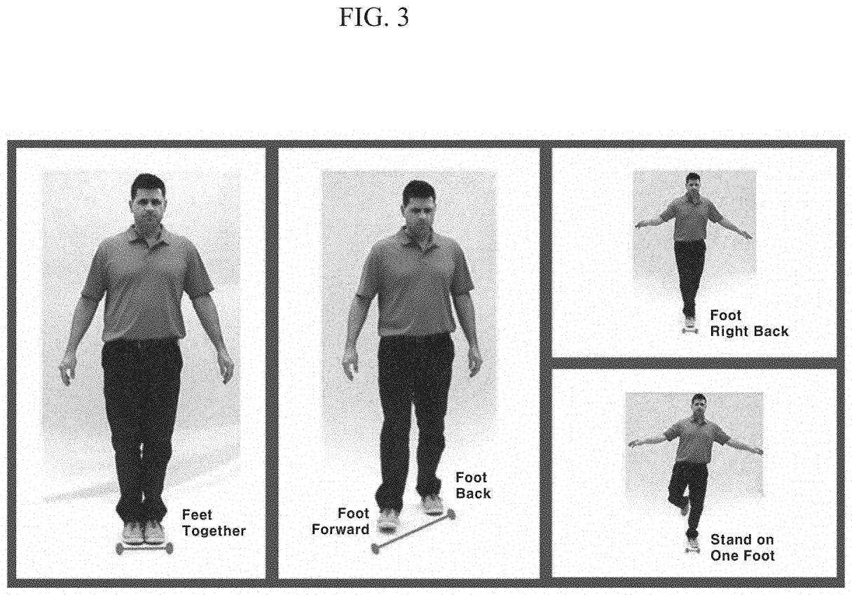

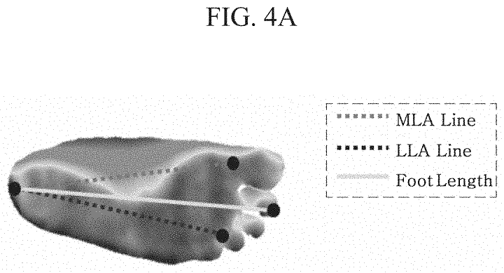

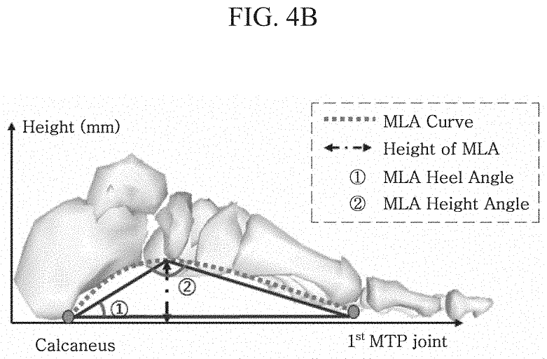

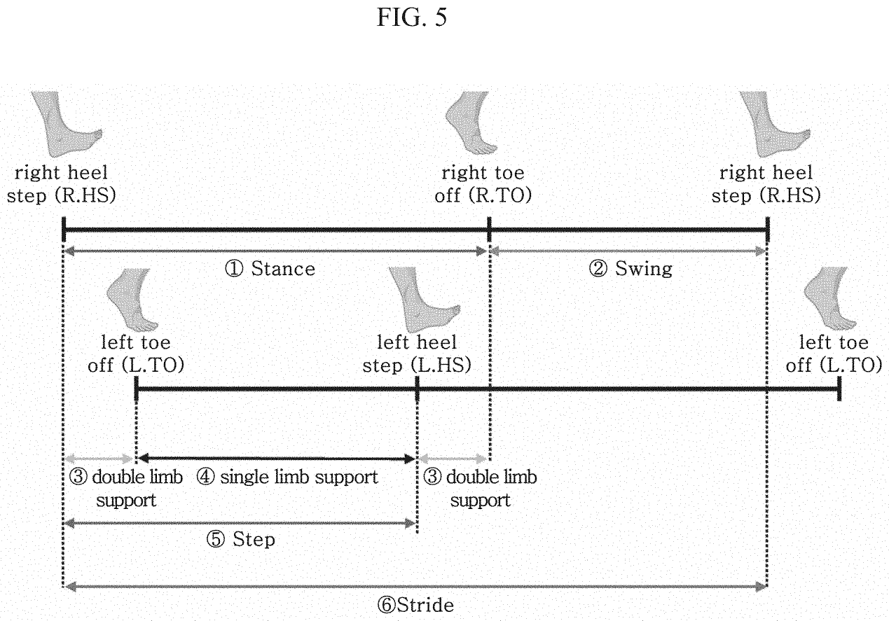

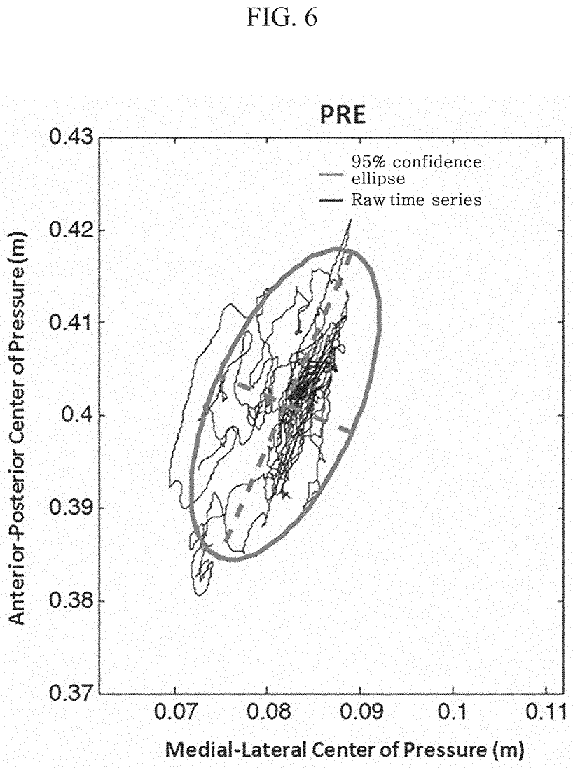

FIG. 1 is a block diagram of a system for predicting geriatric syndrome according to an embodiment of the present invention. FIG. 2 is an exemplary diagram of a data acquisitor. FIG. 3 shows an example of an unstable posture taken by a subject when plantar pressure data is acquired. FIGS. 4A and 4B are exemplary diagrams illustrating a foot depth image and foot characteristic information obtained therefrom. FIG. 5 is an exemplary diagram for explaining gait characteristic information. FIG. 6 is an exemplary diagram illustrating a change in the center of plantar pressure.

Referring to FIGS. 1 to 6, a system for predicting geriatric syndrome 10 includes a data acquisitor 100, a foot characteristic information generator 110, a gait characteristic information generator 120, and a balance characteristic information generator 130, a geriatric syndrome predictor 140 and a database 150.

The system for predicting geriatric syndrome 10 according to the embodiments may be entirely hardware, or may be partly hardware and partly software in one aspect. For example, in the present specification, the system for predicting geriatric syndrome and each component included therein may collectively refer to a device for exchanging data in a specific format and content in an electronic communication method, and software related thereto. As used herein, terms such as “system” or “device” are intended to refer to a combination of hardware and software driven by the hardware. For example, the hardware herein may be a data processing device including a CPU or other processor. In addition, the software driven by hardware may refer to a running process, an object, an executable file, a thread of execution, a program, and the like.

In addition, each component constituting the system for predicting geriatric syndrome is not intended to necessarily refer to physically distinct and separate component. In FIG. 1, although the data acquisitor 100, the foot characteristic information generator 110, the gait characteristic information generator 120, the balance characteristic information generator 130, the geriatric syndrome predictor 140 and the database 150 are shown as separate blocks to be distinguished from each other, this is only functionally dividing the components constituting the system for predicting geriatric syndrome by the operations executed by the corresponding components. Accordingly, according to an embodiment, some or all of the data acquisitor 100, the foot characteristic information generator 110, the gait characteristic information generator 120, the balance characteristic information generator 130, the geriatric syndrome predictor 140 and the database 150 may be integrated in the same single device, or one or more unit may be implemented as separate devices physically separated from other components, and may be components communicatively connected to each other under a distributed computing environment.

The data acquisitor 100 may acquire a depth image and plantar pressure data of a subject's foot. As shown in FIG. 2, the data acquisitor 100 may include a footrest 101, a scanner 102 and a pressure sensor 103, and can acquire the depth image and plantar pressure data of the subject's foot 104 located on the footrest 101. The scanner 102 may be located under the footrest 101 to obtain the depth image of the subject's foot 104 located on the footrest 101. The scanner 102 may photograph toward the sole surface of the subject to be measured located on the footrest 101. The pressure sensor 103 may be located under the four corners of the footrest 101 to obtain a pressure applied to the foot 104 of the subject to be measured located on the footrest 101, that is, plantar pressure. The footrest 101 may be made of a transparent material or an opaque material. The scanner 102 may also include a depth camera or laser, and may be configured to measure the three-dimensional shape and dynamic changes of the subject's foot 104 located on the footrest 101.

The data acquisitor 100 may acquire the foot depth image for generating the foot characteristic information in the foot characteristic information generator 110. Also, the data acquisitor 100 may acquire the plantar pressure data for generating the balance characteristic information in the balance characteristic information generator 130. Here, the foot depth image and the plantar pressure data may be respectively acquired according to different states of stability of the subject's posture. Specifically, the foot depth image may be obtained in a state in which the subject's posture is stable, and the plantar pressure data may be obtained in a state in which the subject's posture is unstable.

The foot depth image may be a depth image for acquiring foot characteristic information including arbitrary information on the shape of each part, overall shape and characteristics of the sole. The foot depth image for generating the foot characteristic information may be obtained while the subject maintains a stable posture on the footrest 101 in order to accurately extract the foot characteristics of the subject. For example, the data acquisitor 100 may acquire foot characteristic information while the subject takes a static and fixed posture. Here, the fixed posture may include at least one of a posture in which the subject to be measured sits with the knee bent at a predetermined angle (e.g., 90 degrees), a posture in which the subject stands on both feet with knees extended, and a posture in which the subject stands on one foot. In some embodiments, the data acquisitor 100 may further include a support member for assisting the subject to easily take a posture.

The plantar pressure data may be data for extracting a center of pressure (CoP) characteristic of a foot. The plantar pressure data may be generated in a state in which the subject is unstable in order to accurately extract the balance characteristics of the subject. Here, the unstable state means a state in which the subject takes a specific posture in order to check the subject's balance ability. The data acquisitor 100 may acquire the plantar pressure data, which is changed to maintain body balance as the subject takes a specific posture, for a predetermined period of time.

As exemplarily shown in FIG. 3, the subject takes an unstable posture on the footrest 101 such as at least one of standing with both feet together with eyes closed, standing with both feet apart more than a predetermined distance or more with eyes closed, standing on one foot with arms wide open, and standing on both feet or one foot while performing mental arithmetic tasks, and the data acquisitor 100 may acquire the plantar pressure data of the subject for a predetermined period of time. That is, if the pressure applied to the user's foot changes in order to maintain balance in an unstable state, the data acquisitor 100 may acquire the plantar pressure data reflecting such pressure change.

The acquired foot depth image may be transmitted to the foot characteristic information generator 110, and the acquired plantar pressure data may be transmitted to the balance characteristic information generator 130, respectively. In addition, the foot depth image, the plantar pressure data, and the data generated by the components to be described later may be stored in the database 150.

The foot characteristic information generator 110 may generate the subject's foot characteristic information from the subject's foot depth image. The foot characteristic information is the arbitrary information related to the sole, and may include arbitrary information on the shape of each part, overall shape and characteristics of the sole. In one embodiment, the foot characteristic information may include a shape of a sole, a width and length of a sole, a height of a foot arch, and an angle of a foot arch curve.

The foot characteristic information generator 110 may extract a medial longitudinal arch (MLA) line and a lateral longitudinal arch (LLA) line from the foot depth image. FIG. 4A is an exemplary diagram illustrating the MLA line, the LLA line, and the like on the foot depth image. Here, the MLA line is a line connecting a heel and a first metatarsal joint. The first metatarsal joint may be the metatarsal joint of the big or index toe in the case of the right foot. The LLA line is a line connecting the heel and a second metatarsal joint. The second metatarsal joint may be the metatarsal joint of the middle or ring finger toe. Here, the heel, the first metatarsal, and the second metatarsal correspond to a portion of the foot skeleton that comes into contact with the ground, and an arch connecting each point may be formed.

FIG. 4B is an exemplary diagram illustrating an MLA curve. The foot characteristic information generator 110 may acquire the foot characteristic information based on at least one of the MLA curve and the LLA curve. The foot characteristic information generator 110 may acquire the foot characteristic information (foot arch height, foot length, sole width, arch curve angle, etc.) as parameters representing the foot characteristics based on the MLA curve, and may acquire the foot characteristic information based on the LLA curve. Each foot characteristic information extracted in one embodiment may be combined according to a predetermined ratio. However, the present invention is not limited thereto, and the foot characteristic information generator 110 may generate the foot characteristic information using the MLA curve or the LLA curve.

The foot characteristic information generator 110 may provide the generated subject's foot characteristic information to the gait characteristic information generator 120 and the geriatric syndrome predictor 140.

The balance characteristic information generator 130 may generate the subject's balance characteristic information from the subject's plantar pressure data. The balance characteristic information may include at least one of a travel distance, a travel speed, a longest reach distance, and an ellipse area of the center of plantar pressure of the subject.

As shown in FIG. 6, the balance characteristic information generator 130 can extract the pressure applied to the footrest by the subject's foot, that is, the center of pressure (CoP) in the anterior/posterior and medial/lateral directions. The plantar pressure data is a measurement of the pressure applied to the foot of the subject taking an unstable posture for a predetermined period of time. The balance characteristic information generator 130 may generate at least one of a travel distance, a travel speed, a longest reach distance and an ellipse area of the plantar pressure center from the plantar pressure data obtained for a predetermined period of time. Here, if the subject's balance ability is good, the travelling of the center of plantar pressure may be small, and if the subject's balance ability is reduced with aging, the travelling of the center of plantar pressure may be large.

The balance characteristic information generator 130 may provide the generated balance characteristic information of the subject to the geriatric syndrome predictor 140.

The gait characteristic information generator 120 may generate the gail characteristic information of the subject based on the foot characteristic information of the subject provided by the foot characteristic information generator 110 by using a first learning model trained to output the gait characteristic information of the subject based on the foot characteristic information.

Here, the gait characteristic information corresponds to a parameter capable of recognizing the gait pattern of the subject. The gait characteristic information may include a temporal parameter and a spatial parameter. As shown in FIG. 5, the gait characteristic information may include, as the temporal parameter, a stride time, a step time, a stance time, a swing time, a single limb support time, a double limb support time, cadence, and the like. In addition, the gait characteristic information may include a stride length, a step length, a gait speed, and the like, as the spatial parameter.

The first learning model according to an embodiment of the present invention may be a machine-trained artificial neural network (ANN) model to output the gait characteristic information based on the input foot characteristic information. The first learning model corresponds to an abstract model that uses the foot characteristic information as an input value and the gait characteristic information of the subject as an output value.

The first learning model may be a model built by deep learning in which a computer performs machine learning to classify objects, which mimics the information processing method of human brain that distinguishes objects after discovering patterns in numerous data. The first learning model may be any one deep learning model among a feedforward neural network model of a multi-layer perceptron structure, a convolutional neural network model that forms a connection pattern between neurons similar to the structure of the visual cortex of an animal, a recurrent neural network model that builds up a neural network at every moment over time, and a restricted Boltzmann machine that can learn a probability distribution for an input set. However, the above-described method is only an example, and the machine learning method according to an embodiment of the present invention is not limited thereto.

The system for predicting geriatric syndrome 10 according to an embodiment of the present invention may further include a first learning model builder 160 to build such a first learning model. The foot characteristic information of a plurality of subjects may be provided as an input value, and the gait characteristic information corresponding thereto may be provided to the first learning model builder 160 as an output value, and a recommendation operation of a machine learning-based improvement process may be performed. An artificial neural network learning can be achieved by adjusting a weight of a connection line between nodes (and adjusting a bias value if necessary) so that a desired output is obtained for a given input. In addition, the artificial neural network may continuously update a weight value through learning. In addition, a method such as back propagation may be used for learning of the artificial neural network, and a first learning model that abstracts a relationship between an input value and an output value may be built. The first learning model builder 160 may be controlled to automatically update the structure of the first learning model for outputting the next gait characteristic information after learning according to a setting.

The built first learning model may be stored in the database 150, and the gait characteristic information generator 120 may generate the gait characteristic information of a new subject by using the built first learning model. The gait characteristic information generator 120 may provide the generated gait characteristic information to the geriatric syndrome predictor 140.

The geriatric syndrome predictor 140 can predict a risk degree of geriatric syndrome of the subject based on the foot characteristic information of the subject, the gait characteristic information of the subject and the balance characteristic information of the subject by using a second learning model trained to output the risk degree of geriatric syndrome based on the foot characteristic information, the gait characteristic information and the balance characteristic information.

The second learning model may be a trained model to predict the risk degree of geriatric syndrome of the subject. The second learning model may predict the risk degree of geriatric syndrome of the subject based on the input foot characteristic information of the subject, the input gait characteristic information of the subject and the input balance characteristic information of the subject. That is, the second learning model may output the risk degree of geriatric syndrome of the subject after receiving the foot characteristic information of the subject (a shape of a sole, a width and length of a sole, a height of a foot arch, an angle of a foot arch curve), the gait characteristic information of the subject (a temporal parameter and spatial parameter that can determine the subject's gait pattern) and the balance characteristic information of the subject (a travel distance, a travel speed, a longest reach distance, and an ellipse area of the center of plantar pressure).

Here, the geriatric syndrome includes frailty, cognitive impairment, sarcopenia and depression, and the second learning model may be a trained model to determine a risk degree for at least one of the frailty, cognitive impairment, sarcopenia and depression of the subject based on the foot characteristic information of the subject, the gait characteristic information of the subject and the balance characteristic information of the subject. The second learning model may be built using various known deep learning structures. The second learning model may be built using a structure such as a convolutional neural network (CNN) and a recurrent neural network (RNN).

The system for predicting geriatric syndrome 10 according to an embodiment of the present invention may further include a second learning model builder 170 for building a second learning model. The second learning model builder 170 may build the second learning model including at least one of a first geriatric syndrome prediction model that determines a degree of frailty of a subject based on an input data, a second geriatric syndrome prediction model that determines a degree of cognitive impairment of a subject based on an input data, a third geriatric syndrome prediction model that determines a degree of muscle loss of a subject based on an input data, and a fourth geriatric syndrome prediction model that determines a degree of depression of a subject based on an input data. The artificial neural network learning can be achieved by adjusting a weight of a connection line between nodes (and adjusting a bias value if necessary) so that a desired output is obtained for a given input. In addition, the artificial neural network may continuously update a weight value through learning. In addition, a method such as back propagation may be used for training the artificial neural network, and the second learning model that abstracts a relationship between an input value and an output value may be built. The second learning model builder 170 may be controlled to automatically update the structure of the second learning model for outputting the next gait characteristic information after learning according to a setting.

Here, the first geriatric syndrome prediction model may be trained to determine a degree of frailty of a subject based on a FRAIL scale or a cardiovascular health study (CHS) frailty index used in a clinical practice. The first geriatric syndrome prediction model is trained to divide and output the degree of frailty of the subject into “non-frailty” and “frailty” or “non-frailty”, “pre-frailty” and “frailty”.

The second geriatric syndrome prediction model may be trained to determine a degree of muscle loss of a subject based on a degree of muscle loss and a degree of muscle function loss evaluated using a body composition meter or a bone density meter, or based on a SARC-F scale. The second geriatric syndrome prediction model may be trained to divide and output the degree of muscle loss of the subject into “non-sarcopenia” and “sarcopenia” according to an input data (foot characteristics, gait characteristics, balance characteristics).

The third geriatric syndrome prediction model may be trained to determine a degree of cognitive impairment of a subject based on a mini-mental state examination (MMSE) or a montreal cognitive assessment (MoCA) score used in a clinical practice. The third geriatric syndrome prediction model is trained to divide and output the degree of cognitive impairment of the subject into “non-cognitive impairment” and “cognitive impairment” or “non-cognitive impairment” and “mild cognitive impairment” and “moderate or higher cognitive impairment”, according to an input data (foot characteristics, gait characteristics, balance characteristics).

The fourth geriatric syndrome prediction model may be trained to determine a degree of depression of a subject based on a geriatric depression scale (GDS) score, etc., used in a clinical practice. The fourth geriatric syndrome prediction model may be trained to divide and output the degree of depression of the subject into “non-depression” and “depression” according to an input data (foot characteristics, gait characteristics, balance characteristics).

In some embodiments, at least one of demographic characteristics (gender, age) and anthropometric characteristics (height, weight, calf circumference) in addition to the foot characteristics, the gait characteristics and the balance characteristics may be further provided as input values, and the second learning model (first to fourth geriatric syndrome prediction models) may be built to predict the subject's geriatric syndrome according to the input data.

The built second learning model may be stored in the database 150, and the geriatric syndrome predictor 140 may predict the risk degree of geriatric syndrome of the subject based on the provided foot characteristic information, gait characteristic information and balance characteristic information of the subject by using the second learning model. That is, the geriatric syndrome predictor 140 may predict the degree of frailty of the subject based on the input data by using the first geriatric syndrome prediction model, the degree of muscle loss of the subject based on the input data by using the second geriatric syndrome prediction model, the degree of cognitive impairment of the subject based on the input data by using the third geriatric syndrome prediction model or the degree of depression of the subject based on the input data by using the fourth geriatric syndrome prediction model.

The geriatric syndrome predictor 140 may individually output results by using each of the first to fourth geriatric syndrome prediction models included in the second learning model. In addition, the geriatric syndrome predictor 140 may comprehensively evaluate the results output from the first to fourth geriatric syndrome prediction models to output the risk degree of geriatric syndrome of the subject. For example, the results of the first to fourth geriatric syndrome prediction models may be digitized and summed, and the geriatric syndrome predictor 140 may provide the subject with a digitized score or grade of the risk degree of geriatric syndrome according to the summed result.

The system for predicting geriatric syndrome according to an embodiment of the present invention can predict a risk degree of geriatric syndrome of a subject only by a test for acquiring simple foot characteristic information and simple balance characteristic information. In other words, it is possible to predict the risk degree of geriatric syndrome and provide it to the subject without expensive equipment, skilled experts, and complicated examination process, and it can provide a subject with an opportunity for early diagnosis of related diseases, management of symptoms, and appropriate treatment.

Hereinafter, a method for predicting geriatric syndrome using foot characteristic information and balance characteristic information according to an embodiment of the present invention will be described.

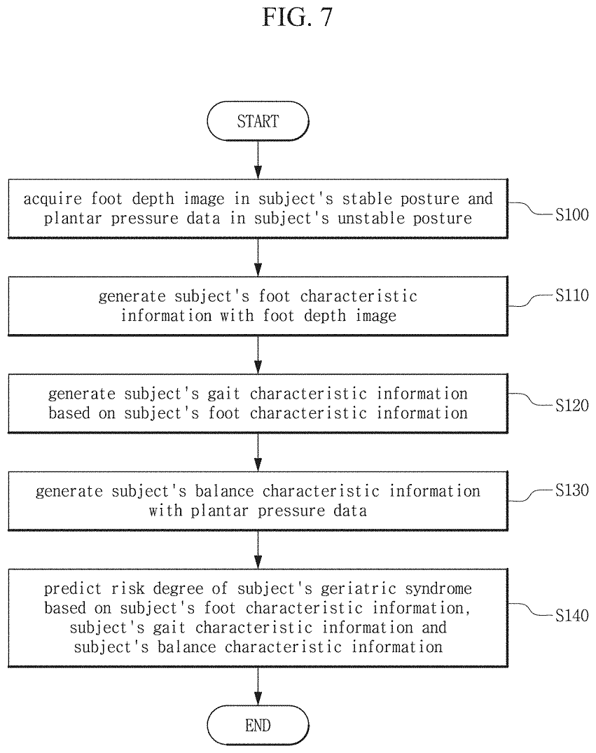

FIG. 7 is a flowchart of a method for predicting geriatric syndrome using foot characteristic information and balance characteristic information according to an embodiment of the present invention. The method may be performed in the system of FIGS. 1 to 6 described above, and FIGS. 1 to 6 may be referred to for explanation in this embodiment.

Referring to FIG. 7, a method for predicting geriatric syndrome according to an embodiment of the present invention includes the steps of acquiring a foot depth image in a state in which a subject's posture is stable, and acquiring plantar pressure data in a state in which the subject's posture is unstable (S100); generating foot characteristic information of the subject with the foot depth image (S110); generating gait characteristic information of the subject based on the subject's foot characteristic information by using a first learning model trained to output the gait characteristic information based on the foot characteristic information (S120); generating balance characteristic information of the subject with the plantar pressure data (S130); and predicting a risk degree of geriatric syndrome of the subject based on the foot characteristic information of the subject, the gait characteristic information of the subject and the balance characteristic information of the subject, by using a second learning model trained to output the risk degree of geriatric syndrome based on the foot characteristic information of the subject, the gait characteristic information of the subject and the balance characteristic information of the subject (S140).

First, a foot depth image is acquired in a state in which the subject's posture is stable, and plantar pressure data is obtained in a state in which the subject's posture is unstable (S100).

This step (S100) may be performed by the data acquisitor 100 of the system for predicting geriatric syndrome 10.

The foot depth image may be a depth image for acquiring the foot characteristic information including arbitrary information on the shape of each part, overall shape and characteristics of the sole. The foot depth image for generating the foot characteristic information may be obtained in a state in which the subject maintains a stable posture on the footrest 101 in order to accurately extract the subject's foot characteristics.

The plantar pressure data may be data for extracting a center of pressure (CoP) characteristic of the foot. The plantar pressure data may be obtained in a state in which the subject is unstable in order to accurately extract the balance characteristics of the subject. Here, the unstable state means a state in which the subject takes a specific posture in order to check the subject's balance ability. The plantar pressure data is a measure of the pressure applied to the subject's foot while the subject takes an unstable posture on the footrest for a predetermined period of time. The unstable posture may correspond to at least one of standing with both feet together with eyes closed, standing with both feet apart more than a predetermined distance with eyes closed, standing on one foot with arms apart, and standing on both feet or one foot while performing mental arithmetic tasks.

Next, the foot characteristic information of the subject is generated with the foot depth image (S110).

This step (S110) may be performed by the foot characteristic information generator 110 of the system for predicting geriatric syndrome 10.

The foot characteristic information is arbitrary information related to the sole, and may include arbitrary information on the shape of each part, overall shape and characteristics of the sole. In one embodiment, the foot characteristic information may include a shape of a sole, a width and length of a sole, a height of a foot arch, and an angle of a foot arch curve. The foot characteristic information generator 110 may extract a medial longitudinal arch (MLA) line and a lateral longitudinal arch (LLA) line from the foot depth image. The foot characteristic information generator 110 may acquire the foot characteristic information based on at least one of the MLA curve and the LLA curve.

Next, using the first learning model trained to output the gait characteristic information based on the foot characteristic information, the gait characteristic information of the subject is generated based on the foot characteristic information of the subject (S120).

This step (S120) may be performed by the gait characteristic information generator 120 of the system for predicting geriatric syndrome 10.

The method according to the present embodiment may further include a step of building a first learning model before performing this step (S120). This step (S120) may be performed using the first learning model trained to output the gait characteristic information based on the foot characteristic information. Here, the first learning model may be a machine trained artificial neural network model to output the gait characteristic information based on the input foot characteristic information.

The gait characteristic information of the subject includes a temporal parameter and a spatial parameter, and the temporal parameters may include at least one of a stride time, a step time, a stance time, a swing time, a single limb support time, a double limb support time and cadence, the spatial parameter may include at least one of a stride length, a step length and a gait speed.

The balance characteristic information of the subject is generated with the plantar pressure data (S130).

This step (S130) may be performed by the balance characteristic information generator 130 of the system for predicting geriatric syndrome 10.

The balance characteristic information generator 130 may determine the center of pressure (CoP) of the foot with the plantar pressure data. The balance characteristic information generator 130 may generate at least one of a travel distance, travel speed, longest reach distance, and ellipse area of the plantar pressure center by tracking the change in the center of pressure of the foot that occurs during a predetermined period of time by using the plantar pressure data. That is, the balance characteristic information may include at least one of a travel distance of the center of plantar pressure, a travel speed of the center of plantar pressure, a longest reach distance of the center of plantar pressure and an ellipse area of the center of plantar pressure. Here, for convenience of description, this step (S130) is described later than the previous steps (S110 and S120), but the order of performing the steps is not limited to the described order. That is, this step (S130) may be performed prior to the previous steps (S110 and S120).

Next, the risk degree of geriatric syndrome is predicted based on the foot characteristic information of the subject, the gait characteristic information of the subject and the balance characteristic information of the subject, by using the second learning model trained to output the risk degree of geriatric syndrome based on the foot characteristic information, the gait characteristic information and the balance characteristic information (S140).

This step (S140) may be performed by the geriatric syndrome predictor 140 of the system for predicting geriatric syndrome 10.

The geriatric syndrome predictor 140 uses the second learning model trained to output the risk degree of geriatric syndrome based on the foot characteristic information, the gait characteristic information and the balance characteristic information to predict the risk degree of geriatric syndrome of the subject based on the foot characteristic information of the subject, the gait characteristic information of the subject and the balance characteristic information of the subject.

The second learning model may be a model trained to predict the risk degree of geriatric syndrome of a subject. The method according to the present embodiment may further include the step of building the second learning model before performing this step (S140).

The second learning model may be a machine-trained artificial neural network model to predict the risk degree of geriatric syndrome based on input foot characteristic information, input gait characteristic information, and input balance characteristic information. In addition, the second learning model may include at least one of the first geriatric syndrome prediction model that determines the degree of frailty of the subject based on the input data, the second geriatric syndrome prediction model that determines the degree of cognitive impairment of the subject based on the input data, the third geriatric syndrome prediction model that determines the degree of muscle loss of the subject based on the input data, and the fourth geriatric syndrome prediction model that determines the degree of depression of the subject based on the input data.

The method for predicting geriatric syndrome according to an embodiment of the present invention can predict a risk degree of geriatric syndrome of a subject only by a test for acquiring simple foot characteristic information and simple balance characteristic information. In other words, it is possible to predict the risk degree of geriatric syndrome and provide it to the subject without expensive equipment, skilled experts, and complicated examination process, and it can provide the subject with an opportunity for early diagnosis of related diseases, management of symptoms, and appropriate treatment.

Although described above with reference to the embodiments, the present invention should not be construed as being limited by these embodiments or drawings, and it will be apparent to those skilled in the art that various modifications and changes can be made in the present invention without departing from the spirit and scope of the present invention as set forth in the claims below.

What is claimed is:

1. A system for predicting geriatric syndrome, comprising:
a data acquisitor that acquires a foot depth image and plantar pressure data of a subject;
a foot characteristic information generator that generates foot characteristic information of the subject with the foot depth image obtained in a state in which the subject's posture is stable;
a gait characteristic information generator that generates gait characteristic information of the subject based on the foot characteristic information of the subject by using a first learning model trained to output the gait characteristic information based on the foot characteristic information;
a balance characteristic information generator that generates balance characteristic information of the subject with the plantar pressure data obtained in a state in which the subject's posture is unstable; and
a geriatric syndrome predictor that predicts a risk degree of geriatric syndrome of the subject based on the foot characteristic information of the subject, the gait characteristic information of the subject and the balance characteristic information of the subject, by using a second learning model trained to output the risk degree of geriatric syndrome based on the foot characteristic information, the gait characteristic information and the balance characteristic information.

2. The system for predicting geriatric syndrome according to claim 1, wherein the foot characteristic information includes at least one of a shape of a sole, a width and length of the sole, a height of a foot arch and an angle of a foot arch curve,
the gait characteristic information of the subject includes a temporal parameter and a spatial parameter,
the temporal parameter includes at least one of a stride time, a step time, a stance time, a swing time, a single limb support time, a double limb support time and cadence,
the spatial parameter includes at least one of a stride length, a step length and a gait speed, and
the balance characteristic information includes at least one of a travel distance of a center of plantar pressure, a travel speed of the center of plantar pressure, a longest reach distance of the center of plantar pressure and an ellipse area of the center of plantar pressure.

3. The system for predicting geriatric syndrome according to claim 1, wherein the second learning model includes at least one of a first geriatric syndrome prediction model that determines a degree of frailty of the subject based on an input data, a second geriatric syndrome prediction model that determines a degree of cognitive impairment of the subject based on the input data, a third geriatric syndrome prediction model that determines a degree of muscle loss of the subject based on the input data, and a fourth geriatric syndrome prediction model that determines a degree of depression of the subject based on the input data.

4. The system for predicting geriatric syndrome according to claim 1, further comprising a database that stores the first learning model and the second learning model.

5. The system for predicting geriatric syndrome according to claim 1, wherein the first learning model is a machine-trained artificial neural network model to output the gait characteristic information based on the input foot characteristic information, and
the second learning model is the machine-trained artificial neural network model to predict the risk degree of geriatric syndrome based on the input foot characteristic information, the input gait characteristic information and the input balance characteristic information.

6. The system for predicting geriatric syndrome according to claim 1, wherein the data acquisitor includes a footrest, a scanner configured to acquire the foot depth image of the subject located on the footrest and a pressure sensor configured to measure the pressure applied to the foot of the subject located on the footrest,
the scanner acquires the foot depth image by photographing the subject's foot for a predetermined period of time while the subject maintains the stable posture on the footrest,
the pressure sensor acquires the plantar pressure data of the subject while the subject maintains the unstable posture on the footrest for a predetermined period of time, and
the unstable posture corresponds to at least one of standing with both feet together with eyes closed, standing with both feet apart more than a predetermined distance with eyes closed, standing on one foot with arms wide open, and standing on both feet or one foot while performing a mental arithmetic task.

7. A method for predicting geriatric syndrome comprising the steps of:
acquiring a foot depth image in a state in which a subject's posture is stable, and plantar pressure data in a state in which the subject's posture is unstable;
generating foot characteristic information of the subject with the foot depth image;
generating gait characteristic information of the subject based on the foot characteristic information of the subject by using a first learning model trained to output the gait characteristic information based on the foot characteristic information;
generating balance characteristic information of the subject with the plantar pressure data; and
predicting a risk degree of geriatric syndrome of the subject based on the foot characteristic information of the subject, the gait characteristic information of the subject and the balance characteristic information of the subject, by using a second learning model trained to output the risk degree of geriatric syndrome based on the foot characteristic information, the gait characteristic information and the balance characteristic information.

8. The method for predicting geriatric syndrome according to claim 7, wherein the foot characteristic information includes at least one of a shape of a sole, a width and length of the sole, a height of a foot arch and an angle of a foot arch curve,
the gait characteristic information of the subject includes a temporal parameter and a spatial parameter,
the temporal parameter includes at least one of a stride time, a step time, a stance time, a swing time, a single limb support time, a double limb support time and cadence,
the spatial parameter includes at least one of a stride length, a step length and a gait speed, and
the balance characteristic information includes at least one of a travel distance of a center of plantar pressure, a travel speed of the center of plantar pressure, a longest reach distance of the center of plantar pressure and an ellipse area of the center of plantar pressure.

9. The method for predicting geriatric syndrome according to claim 7, wherein the second learning model includes at least one of a first geriatric syndrome prediction model that determines a degree of frailty of the subject based on an input data, a second geriatric syndrome prediction model that determines a degree of cognitive impairment of the subject based on the input data, a third geriatric syndrome prediction model that determines a degree of muscle loss of the subject based on the input data, and a fourth geriatric syndrome prediction model that determines a degree of depression of the subject based on the input data.

10. The method for predicting geriatric syndrome according to claim 7, wherein the first learning model is a machine-trained artificial neural network model to output the gait characteristic information based on the input foot characteristic information, and
the second learning model is the machine-trained artificial neural network model to predict the risk degree of geriatric syndrome based on the input foot characteristic information, the input gait characteristic information and the input balance characteristic information.

11. The method for predicting geriatric syndrome according to claim 7, wherein the plantar pressure data is data specific to the pressure applied to the subject's foot while the subject maintains the unstable posture on the footrest for a predetermined period of time, and
the unstable posture corresponds to at least one of standing with both feet together with eyes closed, standing with both feet apart more than a predetermined distance with eyes closed, standing on one foot with arms wide open, and standing on both feet or one foot while performing a mental arithmetic task.

12. The method for predicting geriatric syndrome according to claim 7, further comprising
building the first learning model by using the first learning model trained to output the gait characteristic information based on the foot characteristic information, before performing the step of generating the gait characteristic information of the subject based on the foot characteristic information of the subject; and
building the second learning model by using the second learning model trained to output the risk degree of geriatric syndrome based on the foot characteristic information, the gait characteristic information and the balance characteristic information, before the step of performing the step of predicting the risk degree of geriatric syndrome of the subject based on the foot characteristic information of the subject, the gait characteristic information of the subject and the balance characteristic information of the subject.
